(19) United States
(12) Patent Application Publication
(10) Pub. No.: US 2025/0182346 A1
(43) Pub. Date: Jun. 5, 2025

(54) METHOD AND SYSTEM FOR IMAGE DISPLAY IN A MEDICAL IMAGING SYSTEM

(71) Applicant: WUHAN UNITED IMAGING HEALTHCARE CO., LTD., Wuhan (CN)

(72) Inventors: Jiao BA, Wuhan (CN); Guangdi GUO, Wuhan (CN)

(73) Assignee: WUHAN UNITED IMAGING HEALTHCARE CO., LTD., Wuhan (CN)

(21) Appl. No.: 19/052,261

(22) Filed: Feb. 12, 2025

Related U.S. Application Data
(63) Continuation of application No. PCT/CN2023/134870, filed on Nov. 28, 2023.

(30) Foreign Application Priority Data
Dec. 22, 2022 (CN) 202211667848.0

(51) Int. Cl. G06T 11/00; A61B 8/00; G06F 3/14

(52) U.S. Cl. CPC G06T 11/00; A61B 8/461; A61B 8/565; G06F 3/14

(57) ABSTRACT

Embodiments of the present disclosure provide a method and system for image display in a medical imaging system. The method includes constructing a simulation model corresponding to the imaging device; the simulation model being configured to generate a simulation image corresponding to the imaging device, and the simulation image including simulation image data of the imaging device; generating a module parameter adjustment instruction based on one or more device state parameters transmitted by the imaging device, the module parameter adjustment instruction being configured to adjust one or more parameters of the simulation model; and outputting an adjusted simulation image based on an adjusted simulation model, the adjusted simulation image being used for displaying in the viewing device.

### CROSS-REFERENCE TO RELATED APPLICATION

This application is a continuation of International Application No. PCT/CN2023/134870, filed on Nov. 28, 2023, which claims priority of Chinese Patent Application No. 202211667848.0, filed on Dec. 22, 2022, the contents of each of which is entirely incorporated herein by reference.

### TECHNICAL FIELD

The present disclosure relates to the field of image imaging technology, and in particular relates to a method and system for image display in a medical imaging system.

### BACKGROUND

In a traditional medical image system, an imaging device has a complex structure and is unable to provide timely feedback of image data indicating its operational state, which directly leads to a difficulty for technicians to accurately obtain the operational state of the imaging device when using such imaging device, which is prone to mis-operation and low operating efficiency.

There is therefore a need to provide a method and system for image display in a medical imaging system, which improves the operation efficiency of the imaging device while enabling an operator of the imaging device to be accurately informed of the operational state of the imaging device.

### SUMMARY

One of the embodiments of the present disclosure provides a method for image display in a medical imaging system. The method is implemented on at least one machine, each of which has at least one processor and at least one storage device in a server of the medical image system. The method includes: constructing a simulation model corresponding to the imaging device, the simulation model being configured to generate a simulation image corresponding to the imaging device, and the simulation image including simulation image data of the imaging device; generating a module parameter adjustment instruction based on one or more device state parameters transmitted by the imaging device, the module parameter adjustment instruction being configured to adjust one or more parameters of the simulation model; and outputting an adjusted simulation image based on an adjusted simulation model, the adjusted simulation image being used for displaying in the viewing device.

In some embodiments, the constructing a simulation model corresponding to the imaging device includes: in response to receiving an initialization configuration instruction, constructing the simulation model corresponding to the imaging device.

In some embodiments, the simulation image further includes an identification content corresponding to the simulation image data, the identification content includes at least one of a device operation position, a device state parameter change content, a device operation description, or a next operation suggestion.

In some embodiments, the simulation model includes a plurality of image constructing modules, each of the plurality of image constructing modules is configured with one or more construction parameters, the one or more construction parameters are used for generating the simulation image, and the generating a module parameter adjustment instruction based on one or more device state parameters transmitted by the imaging device includes: generating the module parameter adjustment instruction in a situation where the one or more device state parameters transmitted by the imaging device are inconsistent with the one or more construction parameters corresponding to the each of the plurality of image construction modules; the module parameter adjustment instruction being used for instructing the simulation model to adjust the one or more construction parameters corresponding to the each of the plurality of image construction modules.

In some embodiments, the in response to receiving an initialization configuration instruction, constructing the simulation model corresponding to the imaging device includes: in response to the initialization configuration instruction sent by the imaging device, generating a model initialization configuration parameter, the model initialization configuration parameter being configured for constructing the simulation model corresponding to the imaging device; and generating the simulation image corresponding to the imaging device using the simulation model, the simulation image including the simulation image data of the imaging device, and the simulation image data being used for displaying in the viewing device.

In some embodiments, the one or more device state parameters are outputted by the imaging device based on a digital twin technology.

In some embodiments, the initialization configuration instruction is determined by: determining the initialization configuration instruction in a situation where the imaging device is detected to be switched from a non-operational state to an operational state, the initialization configuration instruction being configured for instructing the server to construct a simulation model corresponding to the imaging device.

In some embodiments, the method further includes: in response to receiving an interactive instruction, outputting simulation image adjustment information, the simulation image adjustment information being configured for instructing the server to adjust the simulation model.

In some embodiments, the method further includes: outputting a device state adjustment parameter based on the simulation image adjustment information, the device state adjustment parameter being configured for instructing the imaging device to make a corresponding operational state adjustment.

In some embodiments, the viewing device includes at least one of a visual reality (VR) device, an augmented reality (AR) device, a holographic imaging device, or a touch display screen; and the viewing device is configured to display the simulation image matching a first display mode based on a received first display mode configuration instruction.

In some embodiments, the simulation image matching the first display mode is displayed by: in a situation where the first display mode is a guidance mode, displaying the simulation image data corresponding to the simulation image transmitted by the server, and the identification content corresponding to the simulation image data; and in a situation where the first display mode is a consultation mode, displaying the simulation image data corresponding to the simulation image transmitted by the server.

In some embodiments, the viewing device is further configured to display the simulation image matching a second display mode based on a received second display mode configuration instruction.

In some embodiments, the displaying the simulation image matching the second display mode is displayed by: in a situation where the second display mode is a default display mode, displaying the simulation image data corresponding to the simulation image transmitted by the server; in a situation where the second display mode is a partial display mode, displaying the simulation image data corresponding to the simulation image transmitted by the server, and displaying, based on a simulation image data selection instruction, a portion of the identification content corresponding to selected simulation image data; and in a situation where the second display mode is a full display mode, displaying the simulation image data corresponding to the simulation image transmitted by the server, and the identification content corresponding to the simulation image data.

In some embodiments, the imaging device is a handheld ultrasound device.

One of the embodiments of the present disclosure provides a method for image display in a medical imaging system. The method is implemented on at least one machine each of which has at least one processor and at least one storage device in a server of a medical image system. The method includes: constructing a simulation model corresponding to the imaging device; generating a simulation image corresponding to the imaging device based on the simulation model, the simulation image including simulation image data of the imaging device; obtaining, in real time, one or more device state parameters of the imaging device; and updating the simulation image in real time based on the one or more device state parameters.

In some embodiments, the constructing a simulation model corresponding to the imaging device includes: in response to receiving an initialization configuration instruction, constructing the simulation model corresponding to the imaging device.

In some embodiments, the simulation image further includes identification content corresponding to the simulation image data, and the identification content includes at least one of a device operation position, a device state parameter change content, a device operation description, or a next operation suggestion.

In some embodiments, the updating the simulation image in real time based on the one or more device state parameters includes: generating, in real time, a module parameter adjustment instruction based on the one or more device state parameters, the module parameter adjustment instruction being used for adjusting one or more parameters of the simulation model; and updating, in real time, the simulation image based on an adjusted simulation model.

One of the embodiments of the present disclosure provides a system for image display in a medical imaging system, including: a construction module configured to construct a simulation model corresponding to an imaging device, the simulation model being configured to generate a simulation image corresponding to the imaging device, and the simulation image including simulation image data of the imaging device; a generation module configured to generate a module parameter adjustment instruction based on one or more device state parameters transmitted by the imaging device, the module parameter adjustment instruction being configured to adjust one or more parameters of the simulation model; and an output module configured to output an adjusted simulation image based on an adjusted simulation model, the adjusted simulation image being used for displaying in a viewing device.

In some embodiments, the construction module is further configured to: in response to receiving an initialization configuration instruction, construct the simulation model corresponding to the imaging device.

In some embodiments, the simulation image further includes identification content corresponding to the simulation image data, and the identification content includes at least one of a device operation position, a device state parameter change content, a device operation description, or a next operation suggestion.

In some embodiments, the simulation model includes a plurality of image constructing modules, each of the plurality of image constructing modules is configured with one or more construction parameters, the one or more construction parameters are used for generating the simulation image, and the generation module is further configured to: generate the module parameter adjustment instruction in a situation where the one or more device state parameters transmitted by the imaging device are inconsistent with the one or more construction parameters corresponding to the each of the plurality of image construction modules; the module parameter adjustment instruction being used for instructing the simulation model to adjust the one or more construction parameters corresponding to the each of the plurality of image construction modules.

In some embodiments, the construction module is further configured to: in response to the initialization configuration instruction sent by the imaging device, generate a model initialization configuration parameter, the model initialization configuration parameter being configured for constructing the simulation model corresponding to the imaging device; and generate the simulation image corresponding to the imaging device using the simulation model, the simulation image including the simulation image data of the imaging device, and the simulation image data being used for displaying in the viewing device.

One of the embodiments of the present disclosure provides a system for image display in a medical imaging system, including: a construction module configured to construct a simulation model corresponding to an imaging device; a simulation image generation module configured to generate a simulation image corresponding to the imaging device based on the simulation model, the simulation image including simulation image data of the imaging device; a real-time obtaining module configured to obtain, in real time, one or more device state parameters of the imaging device; and a real-time updating module configured to update, in real-time, the simulation image based on the one or more device state parameters

One of the embodiments of the present disclosure provides a non-transitory computer-readable storage medium storing computer instructions, when reading the computer instructions in the storage medium, a computer implements the method for image display in a medical imaging system.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1 is a schematic diagram illustrating an application scenario of a system for image display in a medical imaging system according to some embodiments of the present disclosure;

FIG. 2 is a flowchart illustrating an exemplary process for image display in a medical imaging system according to some embodiments of the present disclosure;

FIG. 3 is a schematic diagram illustrating a process for determining one or more current device state parameters based on a first model according to some embodiments of the present disclosure;

FIG. 4 is a flowchart illustrating an exemplary process for generating a simulation image according to some embodiments of the present disclosure;

FIG. 5 is a schematic diagram illustrating a process for determining a simulation model based on a second model according to some embodiments of the present disclosure;

FIG. 6A is a schematic diagram illustrating a simulation image including only simulation image data according to some embodiments of the present disclosure;

FIG. 6B is a schematic diagram illustrating a simulation image including simulation image data and a corresponding identification content according to some embodiments of the present disclosure;

FIG. 7 is a schematic diagram illustrating a state synchronization process for a simulation image of an imaging device according to some embodiments of the present disclosure;

FIG. 8 is a block diagram illustrating an exemplary system for image display in a medical imaging system according to some embodiments of the present disclosure;

FIG. 9 is a block diagram illustrating an exemplary system for image display in a medical imaging system according to some other embodiments of the present disclosure; and

FIG. 10 is a diagram illustrating an internal structure of a computer device according to some embodiments of the present disclosure.

### DETAILED DESCRIPTION

To make the purpose, technical solutions and advantages of the present disclosure more clearly understood, the present disclosure is hereinafter described in further detail in conjunction with the accompanying drawings and embodiments. Unless otherwise defined, all technical and scientific terms used herein have the same meanings as are commonly understood by those skilled in the art falling within the scope of the present disclosure. It should be understood that the specific embodiments described herein are used only to explain the present disclosure, and are not intended to limit the present disclosure.

It should be noted that when an element is considered to be “connected” to another element, it can be either directly connected to the other element or connected to the other element via an intermediate element. In addition, “connecting” in the following embodiments is to be understood as “electrically connecting” or “communicatively connecting”, etc. if there is a transfer of electrical signals or data between the connected objects.

When used here, the singular forms of “one”, “a”, and “the/that” may also include the plural form, unless the context clearly indicates otherwise. It should also be understood that the terms “includes/contains” or “has” etc. designate the presence of the stated feature, whole, step, operation, component, part, or combination thereof, but do not exclude the possibility of the presence or addition of one or more other features, wholes, steps, operations, components, parts, or combinations thereof. Also, as used in the present disclosure, the term “and/or” includes any and all combinations of the items listed.

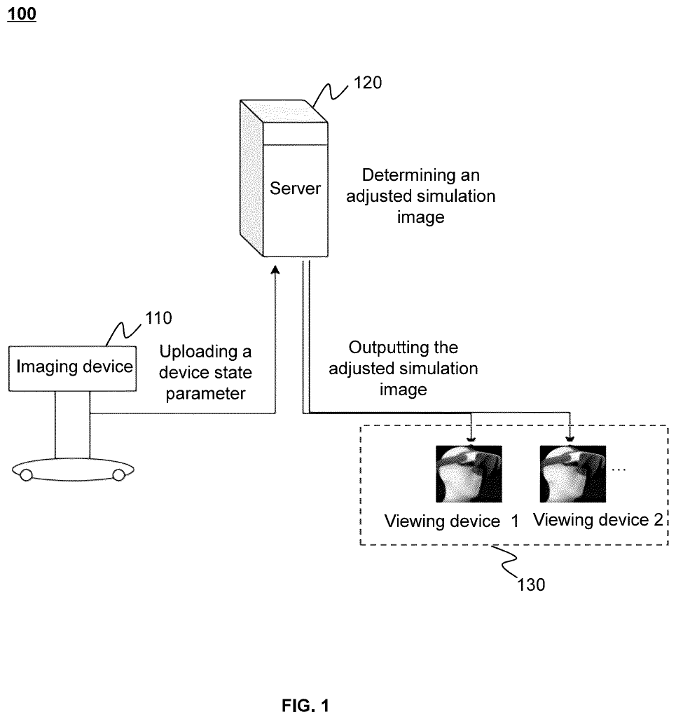

FIG. 1 is a schematic diagram illustrating an application scenario of a system for image display in a medical imaging system according to some embodiments of the present disclosure. In some embodiments, an application scenario of the system 100 for image display in a medical imaging system includes an imaging device 110, a server 120, and a viewing device 130. In some embodiments, the application scenario of the system 100 for image display in a medical imaging system also includes a storage device, a user terminal, and/or a network (not shown in the figure).

The imaging device 110 is used to scan a patient. In some embodiments, the imaging device 110 obtains a medical image of a scanning portion of a scanning object. In some embodiments, the imaging device 110 includes a medical imaging device, a combination of a medical imaging device and an interventional medical device, etc. Exemplary medical imaging devices include a magnetic resonance imaging (MRI) scanner, a positron emission tomography (PET) scanner, a computerized tomography (CT) scanner, a digital radiography (DR) scanner, an ultrasound imaging device, etc., or combinations thereof. Exemplary interventional medical devices include a radiotherapy (RT) device, an ultrasound therapy device, a thermal therapy device, a surgical intervention device, etc., or combinations thereof. In some embodiments, the imaging device 110 includes a handheld ultrasound device.

The server 120 is used to process data related to a method for image display in a medical imaging system. For example, the server 120 constructs a simulation model corresponding to the imaging device. Further, the server 120 generates a module parameter adjustment instruction based on one or more device state parameters transmitted by the imaging device, and outputs an adjusted simulation image based on an adjusted simulation model. In some embodiments, the server 120 is a single server or a server group. The server group is centralized or distributed. In some embodiments, the server 120 is local or remote. In some embodiments, the server 120 is implemented on a cloud platform. Merely way of example, the cloud platform includes a private cloud, a public cloud, a hybrid cloud, a community cloud, a distributed cloud, an internal cloud, a multi-level cloud, etc. or any combination thereof. In some embodiments, the server 120 is integrated or installed on the imaging device 110. In some embodiments, the server 120 is integrated or mounted on the viewing device 130.

The viewing device 130 is used to obtain the simulation image and present it to a user to guide the user. In some embodiments, the viewing device 130 includes a two-dimensional (2D) visual display device, a three-dimensional (3D) visual display device. In some embodiments, the viewing device 130 includes at least one of a virtual reality (VR) device, an augmented reality (AR) device, a mixed reality (MR) device, a hologram device, and a touch display. For example, the viewing device 130 includes a VR helmet, VR glasses, a VR eyepiece, an AR helmet, AR glasses, an AR eyepiece, etc., or any combination of the above. For another example, the viewing device 130 includes a display of the handheld ultrasound device.

In some embodiments, there are one or more viewing devices 130, a specific count of which is determined based on an actual application requirement. When there are a plurality of viewing devices 130, requirements of a remote guidance and a remote consultation are satisfied.

In some embodiments, the application scenario of the system 100 for image display in a medical imaging system further includes one or more other devices, for example, a storage device, a user terminal, and/or a network.

In some embodiments, the imaging device and/or the viewing device is a handheld ultrasound device. The handheld ultrasound device refers to a portable, handheld diagnostic ultrasound device.

The storage device stores data, instructions, and/or any other information. In some embodiments, the storage device stores the data and/or instructions related to a method for image display in a medical imaging system. For example, the storage device stores simulation image data. For another example, the storage device stores instructions for generating the module parameter adjustment instruction based on one or more device state parameters transmitted by the imaging device.

In some embodiments, the storage device is connected to the network to communicate with one or more other components (e.g., the imaging device 110, the server 120, the viewing device 130, and/or the storage device, etc.) of the application scenario of the system 100 for image display in a medical imaging system. The one or more components of the application scenario of the system 100 for image display in a medical imaging system access the data or instructions stored in the storage device through the network. In some embodiments, the storage device is a part of the server 120.

The storage device stores data to be processed by the server 120, which is either integrated on the server 120 or placed on a cloud or other network servers.

In some embodiments, the user interacts with a system for image display in a medical imaging system through the user terminal. For example, the user inputs a first display mode and/or a second display mode through the user terminal. Exemplarily, the user terminal includes a mobile device, a tablet, a laptop, etc., or any combination thereof.

The network includes any suitable wired or wireless network that facilitates an exchange of the information and/or data. For example, the server 120 and the imaging device 110 passes the information and/or data through Bluetooth and/or a network. For another example, the imaging device 110 and the viewing device 130 communicates with the server 120 through the Bluetooth and/or the network.

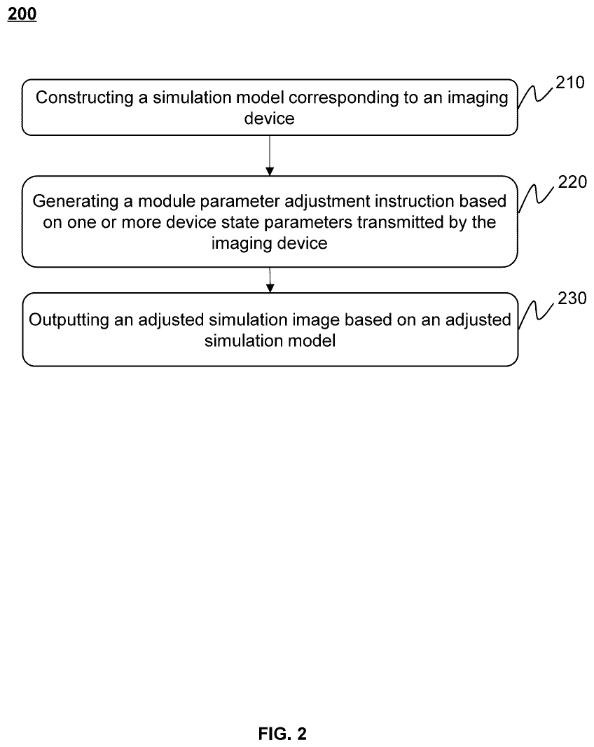

FIG. 2 is a flowchart illustrating an exemplary process for image display in a medical imaging system according to some embodiments of the present disclosure. As shown in FIG. 2, process 200 includes one or more of the following operations. In some embodiments, one or more of the operations of the process 200 shown in FIG. 2 are implemented in the application scenario of the system 100 for image display in a medical imaging system shown in FIG. 1. For example, the process 200 shown in FIG. 2 is stored in a storage device in a form of instructions and called and/or executed by the server 120.

In 210, a simulation model corresponding to an imaging device may be constructed.

A simulation model refers to a virtual model used to reflect the imaging device.

In some embodiments, the server constructs the simulation model based on a digital twins (DT) technology. For example, the server constructs the simulation model through the following three steps: \textcircled{1} establishing a physical model, the physical model being a mathematical model of the imaging device that describes a form, a feature, and an operating rule of the imaging device. When establishing the physical model, various factors of the imaging device, including a size, a material, and an operation mechanism need to be considered. By establishing the physical model, an operational state of the imaging device is initially simulated. \textcircled{2} Data collection and processing, in which the data collection refers to various data collected from the imaging device, including a tube voltage, a tube current, an exposure time and other operation parameters. The data processing, on the other hand, is to clean, analyze, and convert the collected data so that the collected data may be used by the simulation model. Through the data collection and processing, a comprehensive perception and monitoring of the imaging device can be realized. \textcircled{3} Establishing the simulation model, i.e., a third operation of the simulation model performed to establish the simulation model, which involves combining the physical model and the collected data to build the simulation model through tools such as modeling software. The establishment of the simulation model needs to consider accuracy and precision of the physical model and the data, and at the same time ensure stability and reliability of the model.

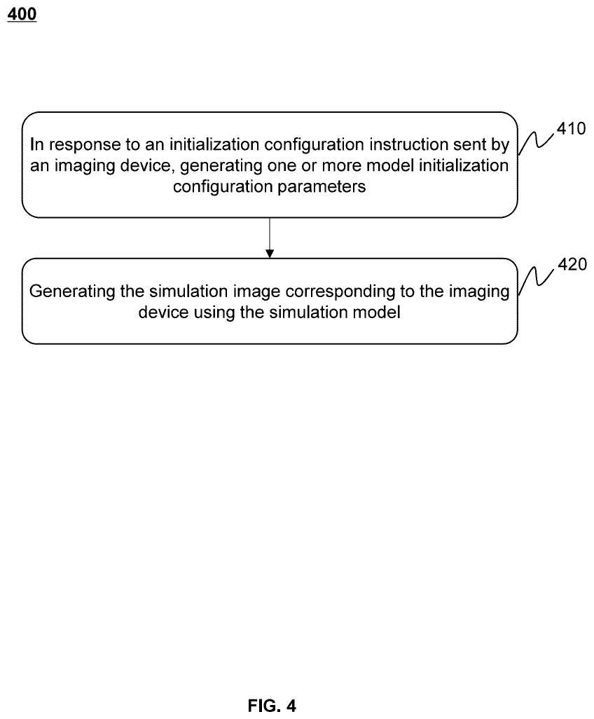

In some embodiments, in response to receiving an initialization configuration instruction, the server constructs a simulation model corresponding to the imaging device. Some other embodiments of constructing the simulation model corresponding to the imaging device in response to receiving the initialization configuration instruction may be seen in descriptions in FIG. 4.

In some embodiments, the server numbers the imaging devices and pre-construct the simulation models corresponding to each imaging device, then stores numberings of the imaging devices and the corresponding simulation models in the storage device. When an image display of the medical image system is required, the server calls the corresponding simulation model in the storage device, and uses the simulation model directly as the simulation model corresponding to the constructed imaging device.

In some embodiments, in response to receiving instruction information input by a user, the server constructs the simulation model corresponding to the imaging device. The instruction information refers to information indicating that the image display is required. For example, the user who believes that the image display is required during the use of the imaging device enters the instruction information to instruct the server to display the simulation image.

In some embodiments, in response to satisfying a preset condition, the server constructs a simulation model corresponding to the imaging device. The preset condition refers to a preset condition that automatically triggers the image display. For example, the preset condition includes that a user operation interval time exceeding an interval time threshold, a user operation step does not comply with a conventional operation step, an operation parameter of the user input not being within a corresponding operation parameter range, etc. When the user operation interval time exceeds the interval time threshold, the user operation step does not comply with the conventional operation step, and/or the operation parameter the user inputs is not in the corresponding operation parameter range, some problems occur. At this time, a construction of the simulation image is triggered to display simulation image data to the user through a viewing device, thereby helping the user to better operate the imaging device.

In some embodiments, the simulation model is used to generate the simulation image corresponding to the imaging device.

The simulation image refers to a 2D or 3D image that includes the imaging device. In some embodiments, the simulation image includes the simulation image data of the imaging device.

The simulation image data refers to the simulation image data for indicating an operational state of the imaging device. For example, the simulation image data includes the simulation image data corresponding to a host, a probe, and a monitor, respectively, in the imaging device.

In some embodiments, the simulation image further includes an identification content corresponding to the simulation image data.

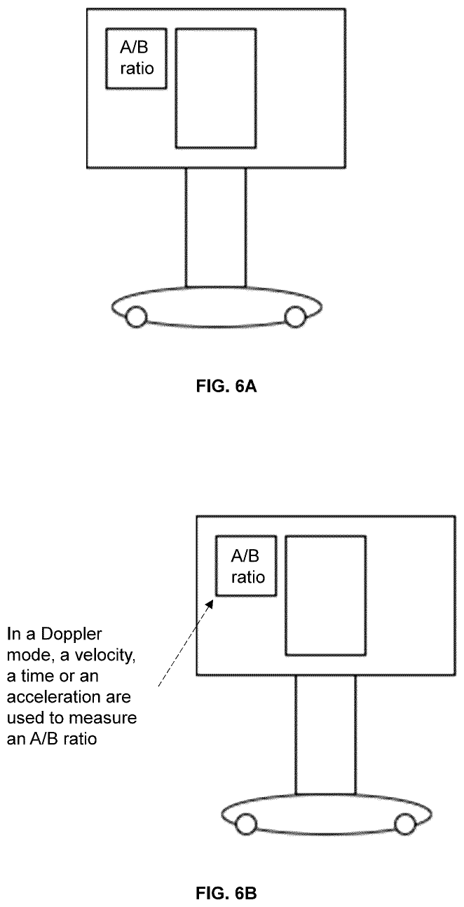

The identification content corresponding to the simulation image data refers to an identification content that matches the operational state of the imaging device indicated by the simulation image data, and a specific content of the identification content may be a specific description for the operational state of the imaging device indicated by the simulation image data. In some embodiments, the identification content corresponding to the simulation image data includes at least one of a device operation position, a device state parameter change content, a device operation description, or a next operation suggestion. The device operation position refers to an operation position of the imaging device at a current moment; the device state parameter change content refers to a changed content of the device state parameter at the current moment compared to the device state parameter at a historical moment; the device operation description refers to a description of the operation of the device at the current moment; the next operation suggestion refers to an operation suggestion for a future moment. More contents on the device state parameter may be seen in the descriptions below. For example, as shown in FIG. 6A, the simulation image includes only the simulation image data. For example, as shown in FIG. 6B, the simulation image includes the simulation image data, as well as the identification content of the simulation image data (e.g., “In a Doppler mode, a velocity, a time or an acceleration are used to measure an A/B ratio”).

In some embodiments of the present disclosure, a real-time operation guidance is provided to the user by displaying the simulation image including the identification content corresponding to the simulation image data in front of user's eyes through the viewing device. This enables the user to obtain in real time the current device operation position, the device state parameter change content, the device operation instruction, the next operation suggestion, etc., to help the user better operate the imaging device, with a strong interactivity.

In 220, a module parameter adjustment instruction may be generated based on one or more device state parameters transmitted by the imaging device.

The one or more device state parameters may be transmitted by the imaging device and used to indicate its current operation state. In some embodiments, the device state parameter(s) include various working parameters used to indicate the operational state of various parts in the imaging device. For example, the one or more device state parameters include one or more parameters indicating the image data generated by the imaging device, one or more adjusted parameters on a touch screen of the imaging device (which include one or more parameter names and corresponding parameter values of the one or more adjusted parameters), one or more parameter values corresponding to an adjusted button or knob in the imaging device (which include a button or knob name and the parameter values indicating the button or the knob under the corresponding operational state), one or more state parameters corresponding to an operational state indicator of the imaging device (which are expressed as one or more parameter values indicating the corresponding operational state of the operational state indicator).

In some embodiments, the imaging device outputs the one or more device state parameters to the server based on a digital twin technology. For example, the imaging device digitizes its device state parameters and transmits the device state parameters to the server through a network.

The module parameter adjustment instruction refers to a module parameter adjustment instruction used to instruct the server to make corresponding parameter adjustments to the simulation model. For example, the module parameter adjustment instruction includes: in a situation where there is an inconsistency between the one or more device state parameters and one or more construction parameters stored by one or more image construction modules in the simulation model, the server generates the module parameter adjustment instruction by processing the one or more device state parameters.

In some embodiments, when the simulation image includes the simulation image data and the corresponding identification content, the module parameter adjustment instruction may indicate simultaneous adjusting for the simulation image data and the corresponding identification content, or may indicate independent adjusting for any one of the simulation image data or the identification content corresponding to the simulation image data.

In some embodiments, the simulation model includes a plurality of image construction modules. Each image construction module is configured with one or more corresponding construction parameters used to generate the simulation image. By processing the one or more device state parameters transmitted by the imaging device, the server generates the module parameter adjustment instruction when the one or more device state parameters transmitted by the imaging device are inconsistent with the corresponding construction parameters of each image construction module. The above module parameter adjustment instruction is used to instruct the simulation model to adjust the one or more construction parameters corresponding to the image construction module.

The image construction modules refer to modules obtained by dividing the simulation model based on a preset rule. For example, the image construction modules may include modules corresponding to parts including a host computer, one or more keys, a probe, a trackball, and a display of the above-described imaging device. For another example, the image construction modules may include modules obtained by dividing the simulation model based on region(s).

In some embodiments, each image construction module is configured with one or more corresponding construction parameters.

A construction parameter may correspond to one of the image construction modules in the simulation model. In some embodiments, the one or more construction parameters are used to generate the simulation image.

In some embodiments, if the simulation image includes the simulation image data, the server corresponds the simulation image data to different construction parameters, respectively. For example, the server uses a plurality of image construction modules with different construction parameters to respectively store a plurality of construction parameters for generating the simulation image data. In some embodiments, if the simulation image includes the simulation image data and the identification content, the server corresponds the simulation image data and the identification content to different construction parameters, respectively. For example, the server uses a plurality of image construction modules with different construction parameters to store the plurality of construction parameters for generating the simulation image data, and a plurality of construction parameters for generating the identification content, respectively.

In some embodiments, the server analyzes whether the one or more device state parameters are consistent with the one or more construction parameters corresponding to the respective image construction module. In response to a situation in which there is an inconsistency between the one or more device state parameters and one or more construction parameters stored by some image construction modules in the simulation model, the server generates the module parameter adjustment instruction by processing the one or more device state parameters.

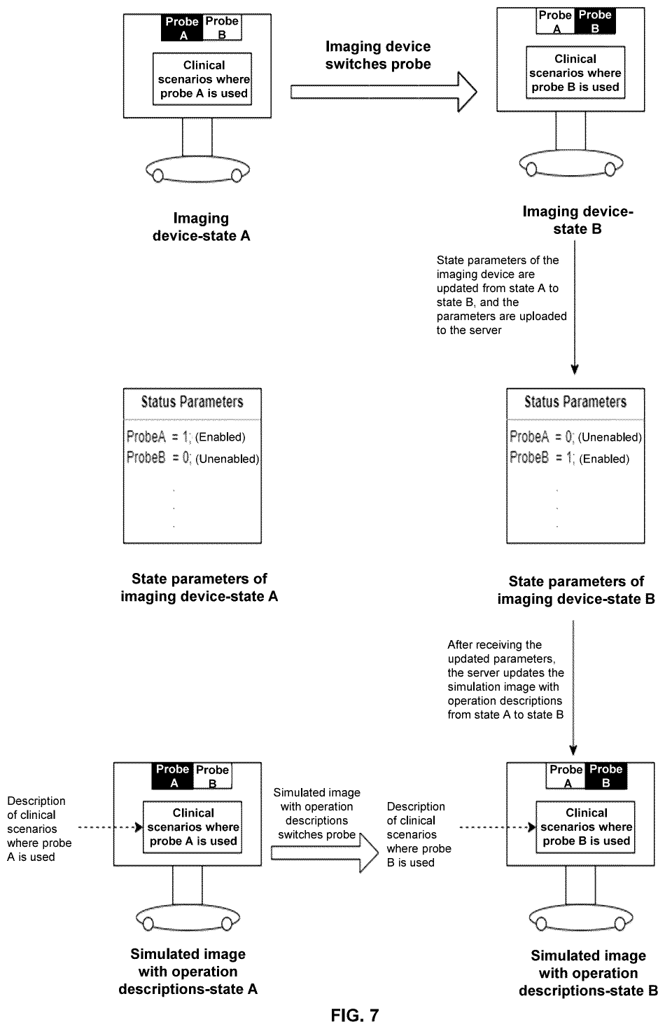

Exemplarily, as shown in FIG. 7, assuming that in the one or more device state parameters, an enabled state is indicated by using an operational state parameter 1, and an unenabled state is indicated by using an operational state parameter 0, then when the imaging device switches an enabled probe, the server generates a corresponding module parameter adjustment instruction based on the one or more device state parameters transmitted by the imaging device. The corresponding module parameter adjustment instruction indicates a corresponding parameter adjustment of the simulation model corresponding to the imaging device by adjusting the operational state parameter corresponding to a probe A from 1 to 0 and adjusting the operational state parameter corresponding to a probe B from 0 to 1. It should be noted that in FIG. 7, the “Probe A” represents the probe A, the “Probe B” represents the probe B, and “Status Parameters” represent the operational state parameters.

In some embodiments, if the quality of a network transmission at a current moment is not good, or when the data transmission is interrupted, the current device state parameter(s) at the current moment are unable to be received. At this time, the current device state parameter(s) transmitted by the imaging device is predicted based on a device state parameter sequence transmitted by the imaging device received during a preset period of time; and, in a situation where the one or more device state parameters transmitted by the imaging device are inconsistent with the one or more construction parameters corresponding to the plurality of image construction modules, the current device state parameter(s) are processed and a module parameter adjustment instruction is generated.

The preset period of time refers to a time period set in advance. For example, the preset period of time is 10 min, 5 min, 2 min, etc. In some embodiments, the preset period of time is set artificially.

The device state parameter sequence refers to a sequence of several device state parameters arranged in a chronological order over a preset period of time. A count of the plurality of device state parameters, and time point(s) at which the plurality of state parameters are located may be set artificially.

A current device state parameter refers to a device state parameter at the current moment.

In some embodiments, the server obtains its current device state parameter(s) by vector matching. For example, a database includes a plurality of reference vectors, and there are corresponding current device state parameter(s) for each reference vector in the plurality of reference vectors. The plurality of reference vectors are constructed based on a historical device state parameter sequence. The server constructs to-be-matched vectors based on the device state parameter sequence, calculates a distance between each of the reference vectors and each of the to-be-matched vectors, respectively, and determines a reference vector whose distance from the to-be-matched vector satisfies a preset distance condition as a target vector, and determines the current device state parameter corresponding to the target vector as the current device state parameter corresponding to the vector to be matched. The preset distance condition may be set based on an actual situation. For example, the preset distance condition is that a vector distance is less than a distance threshold, etc.

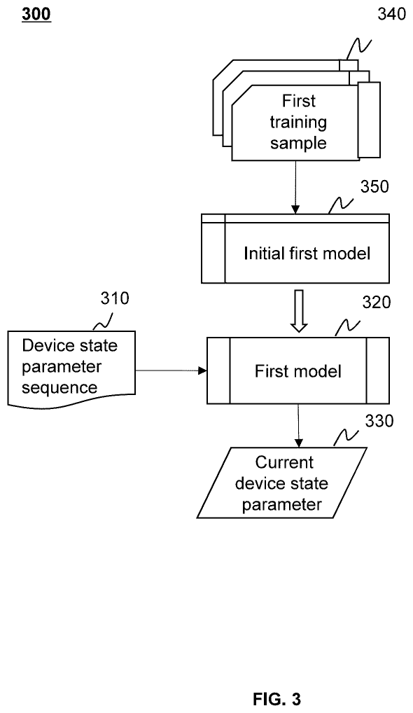

In some embodiments, as shown in FIG. 3, which is a schematic diagram illustrating a process 300 for determining one or more current device state parameters based on a first model according to some embodiments of the present disclosure, the server determines, based on a device state parameter sequence 310, one or more current device state parameters 330 based on a first model 320.

In some embodiments, the first model 320 is a machine learning model. For example, the first model 320 includes one or a combination of a convolutional neural networks (CNN) model, a deep neural networks (DNN) model, etc.

In some embodiments, an input of the first model 320 includes the device state parameter sequence 310, and an output includes the one or more current device state parameters 330.

In some embodiments, the first model 320 is obtained by training based on a large number of first training samples 340 with first labels. In some embodiments, the server inputs the plurality of first training samples 340 into an initial first model 350, updates one or more parameters of the initial first model 350 through training iterations until a trained model satisfies a preset termination condition, and obtains a trained first model 320. The preset termination condition is that a loss function is less than a threshold or converges, or a training iteration count reaches a threshold. In some embodiments, a method of iteratively updating the parameters of the model includes a model training method such as a random gradient descent method.

In some embodiments, the first training sample 340 includes one or more sample device state parameters between a first historical time point and a second historical time point obtained from historical data. The first historical time point precedes the second historical time point, and a duration between the first historical time point and the second historical time point is a preset time period. The one or more first labels are actual device state parameters at a third historical time point, and the third historical time point is after the second historical time point.

In some embodiments of the present disclosure, the first model allows for a fast and accurate prediction of the one or more current device state parameters, which in turn allows for generating a module parameter adjustment instruction based on the predicted current device state parameters when a network transmission quality is poor (or even when the data transmission is interrupted), so as to ensure that an image display is not damaged.

In some embodiments, the server adjusts corresponding construction parameters of an image construction module based on the module parameter adjustment instruction. For example, if in the received one or more device state parameters (understandably, when the network transmission quality is poor, the received one or more device state parameters are predicted current device state parameters), the parameters of the image data indicating the imaging device are inconsistent with the parameters of the image data stored in some of the image construction modules, the parameters of the received image data are adopted to replace the parameters of the image data currently stored in the corresponding image construction module. For example, if in the received one or more device state parameters, some other parameters indicating the operational state of the imaging device are inconsistent with the other parameters stored in some of the image construction modules, then the other parameters received are adopted to replace the other parameters currently stored in the corresponding image construction modules.

In some embodiments of the present disclosure, by means of adjusting the corresponding parameters of each image construction module in the simulation model corresponding to the imaging device based on the one or more device state parameters transmitted by the imaging device, a data consistency between the operational state of the imaging device indicated by the simulation image corresponding to the imaging device and the actual operational state of the imaging device is ensured, thereby ensuring that a user of the viewing device is able to accurately informed of the operational state of the imaging device. And, by dividing the simulation model into a plurality of image construction modules and analyzing the consistency between the one or more device state parameters and the one or more construction parameters corresponding to the image construction modules, and in case of inconsistency, adjustments may be made to the inconsistent image construction modules without having to adjust all the modules, which reduces a calculation pressure on the server when processing data and improves the efficiency.

In 230, an adjusted simulation image may be output based on an adjusted simulation model.

The adjusted simulation model refers to the simulation model corresponding to the adjusted imaging device. 20

The adjusted simulation image refers to the adjusted simulation image output by the server based on the adjusted simulation model. In some embodiments, the adjusted simulation image includes the adjusted simulation image data of the imaging device.

The adjusted simulation image data refers to simulation image data used to indicate the operational state of the adjusted imaging device. After adjustment, the operational state of the imaging device indicated by the simulation image data is consistent with an actual operational state of the imaging device.

In some embodiments, the server also outputs an adjusted identification content based on the adjusted simulation model. The adjusted identification content refers to the identification content that matches the operational state of the imaging device indicated by the adjusted simulation image data.

In practical application, the adjusted simulation image data and the adjusted identification content may either be displayed together in the above-described viewing device, i.e., the adjusted simulation image data and the adjusted identification content are displayed at the same time by the above-described viewing device, or only the adjusted simulation image data may be displayed, i.e., only the adjusted simulation image data is displayed by the above-described viewing device.

The adjusted simulation image is used for displaying in the viewing device.

In some embodiments, the server determines the adjusted simulation image based on the one or more construction parameters corresponding to the image construction module(s) in the adjusted simulation model, thereby transmitting the adjusted simulation image to the viewing device for displaying the simulation image including the adjusted simulation image data and/or the adjusted identification content.

In some embodiments, the viewing device includes at least one of a VR device, an AR device, a holographic imaging device, or a touch display screen. Some other embodiments of a viewing device can be found in related descriptions in FIG. 1.

In some embodiments, the viewing device displays the simulation image matching a first display mode based on a received first display mode configuration instruction.

The first display mode refers to display modes that indicate different uses of the displayed image. For example, the first display mode includes a guidance mode and a consultation mode.

In some embodiments, the user selects the first display mode by one or more adjustment buttons on the viewing device according to different requirements. In some embodiments, the user selects the first display mode according to different requirements through a user terminal.

In some embodiments, in a situation where the first display mode is a guidance mode, the viewing device displays the simulation image data corresponding to the simulation image transmitted by the server, and the identification content corresponding to the simulation image data. This mode helps the user of the viewing device to perform the corresponding device operation based on the current operational state of the imaging device, to avoid problems of mis-operation(s) and a lower operational efficiency due to a difficulty of accurately being informed of the operational state of the imaging device.

In some embodiments, in a situation where the first display mode is a consultation mode, the viewing device displays the simulation image data corresponding to the simulation image transmitted by the server. The consultation mode only displays the simulation image data corresponding to the simulation image transmitted by the server, which enables the user of the viewing device to perform the corresponding collaborative operation or the corresponding guidance operation in real time based on the operational state of the imaging device in a process of a remote collaboration or a remote consultation, so as to improve an efficiency of the remote collaboration or the remote consultation by improving a real-time data exchange in the process of the remote collaboration or the remote consultation.

In some embodiments, the viewing device also displays the simulation image that matches a second display mode based on a received second display mode configuration instruction.

The second display mode refers to a display mode that indicates a different display content of the displayed image. For example, the second display mode includes a default display mode, a partial display mode, and a full display mode.

A manner of adjusting the second display mode is similar to the manner of adjusting the first display mode, which is not repeated here.

In some embodiments, in a situation where the second display mode is the default display mode, the viewing device displays the simulation image data corresponding to the simulation image transmitted by the server. In the default display mode, the simulation image data corresponding to the simulation image transmitted by the server is displayed, i.e., the identification content corresponding to the simulation image data is hidden in the default display mode. The default display mode may correspond to a low display accuracy level of the viewing device.

In some embodiments, in a situation where the second display mode is the partial display mode, the observing device displays the simulation image data corresponding to the simulation image transmitted by the server, and displays, based on a simulation image data selection instruction, a portion of the identification content corresponding to selected simulation image data. The partial display mode displays the simulation image data corresponding to the simulation image transmitted by the server, and displays the identification content corresponding to the simulation image data of a corresponding portion of the simulation image data based on the received simulation image data selection instruction. That is, in the partial display mode, only the identification content corresponding to the selected portion of data in the simulation image data is displayed. The partial display mode corresponds to a middle display accuracy level of the viewing device. The simulation image data selection instruction is a data selection instruction generated for instructing the viewing device to display an operation description on the corresponding portion based on a simulation image data selection operation of the user in the viewing device (i.e., the user selects the corresponding portion by mouse clicking, touching the screen, etc., in the viewing device that displays the simulation image data).

In some embodiments, in a situation where the second display mode is the full display mode, the viewing device displays the simulation image data corresponding to the simulation image transmitted by the server, and the identification content corresponding to the simulation image data. In the full display mode, the simulation image data corresponding to the simulation image transmitted by the server, and the identification content corresponding to the simulation image data is displayed, which means, in the full display mode, the simulation image data that indicates the operational state of the imaging device, as well as identification content matching the operational state of the imaging device indicated by the simulation image data, are completely displayed. The full display mode may correspond to a high display accuracy level of the viewing device.

It is noted that the first display mode and the second display mode are applied either separately or simultaneously in the viewing device, i.e., in the viewing device, only either one of the first display mode and the second display mode is applied, or both the first display mode and the second display mode are applied in the viewing device.

In some embodiments of the present disclosure, by constructing the simulation model with a real-time data synchronization function for generating the simulation image indicating the current operational state of the imaging device by adopting a digital twin technology between the server and the imaging device, the real-time display of the simulation image data indicating the current operational state of the imaging device as well as the identification content corresponding to the simulation image data in the viewing device are implemented, which not only solves the problem that an operator of the imaging device is prone to mis-operation due to the difficulty of accurately knowing the operational state of the imaging device, but also effectively improves the operation efficiency of the imaging device.

It should be noted that the foregoing description of the process of the method for image display in a medical imaging system is for the purpose of example and illustration only, and does not limit the scope of application of the present disclosure. For those skilled in the art, various corrections and changes are made to the process of the method for image display in a medical imaging system under the guidance of the present disclosure. However, these corrections and changes remain within the scope of the present disclosure.

In some embodiments, the medical image system also includes a voice device. The adjusted identification content is broadcast by the voice device.

The voice device is used to broadcast the adjusted identification content. In some embodiments, the voice device includes a moving coil microphone, a condenser microphone, a ribbon microphone, etc.

By using the voice device, when the user is unable to carefully refer to the adjusted identification content displayed in the viewing device, the user is reminded of the adjusted identification content by broadcasting the adjusted identification content through the voice device in a timely manner.

In some embodiments, in response to receiving interactive instruction(s), the server outputs simulation image adjustment information. The simulation image adjustment information is used to instruct the server to adjust the simulation model accordingly.

An interaction instruction refers to an instruction originating from the user and is used to make corresponding adjustments to the simulation image generated by the simulation model.

In some embodiments, the user enters the interaction instruction through the viewing device or the user terminal.

The simulation image adjustment information refers to information used to instruct the server to adjust the simulation model accordingly. For example, the simulation image adjustment information is a parameter value used to make corresponding adjustments to the operational state of the imaging device indicated by the simulation image generated by the simulation model.

In some embodiments, the server also outputs one or more device state adjustment parameters based on the simulation image adjustment information. The one or more device state adjustment parameters are configured for instructing the imaging device to make a corresponding operational state adjustment.

The device state adjustment parameter refers to a parameter that is used to instruct the imaging device to make an appropriate operational state adjustment. The imaging device adjusts the operational state to a state that is consistent with the operational state of the imaging device as indicated by the simulation image generated by the simulation model in the server described above based on the one or more device state adjustment parameters.

It is understood that the outputting the simulation image adjustment information, and the outputting the one or more device state adjustment parameters, may be simultaneous. That is, the server may send the simulation image adjustment information to the simulation model to instruct the simulation model to make corresponding state adjustments to the simulation image generated by the simulation model; and, at the same time, the server may send the one or more device state adjustment parameters output based on the simulation image adjustment information to the imaging device to instruct the imaging device to make corresponding operational state adjustments.

Exemplarily, assuming that the viewing device receives an interaction instruction of pressing a button A in the simulation image, the viewing device uploads the interaction instruction to the server through a network. The server may output the simulation image adjustment information to adjust the corresponding simulation model of the imaging device accordingly, and thereby adjusting the simulation image generated by the simulation model to the simulation image of the corresponding state of pressing the button A down. After that, the server may also output one or more device state adjustment parameters based on the simulation image adjustment information to instruct the imaging device to similarly adjust the operational state of the imaging device to the corresponding state of pressing the button A down.

In some embodiments of the present disclosure, by performing corresponding adjustment on the simulation image generated by the simulation model corresponding to the imaging device and the actual operational state of the imaging device based on the interactive instruction given by the user, not only the data consistency between the operational state of the imaging device indicated by the simulation image corresponding to the imaging device and the actual operational state of the imaging device is ensured, but also effectively improves the operational efficiency of the imaging device.

FIG. 4 is a flowchart illustrating an exemplary process for generating a simulation image according to some embodiments of the present disclosure. As shown in FIG. 4, a process 400 includes one or more of the following operations. In some embodiments, one or more of the operations of the process 400 as shown in FIG. 4 are realized in the application scenario of the system 100 for image display in a medical imaging system shown in FIG. 1. For example, the process 400 shown in FIG. 4 is stored in as instructions in a storage device and called and/or executed by the server 120.

In 410, in response to an initialization configuration instruction sent by an imaging device, one or more model initialization configuration parameters may be generated.

The initialization configuration instruction refers to an instruction used to instruct the server to construct a simulation model corresponding to the imaging device. Based on the instruction, the server constructs the simulation model corresponding to the imaging device.

In some embodiments, upon detecting a switch of the imaging device from a non-operational state to an operational state, the server processes one or more initial device state parameters to determine the initialization configuration instruction; the initialization configuration instruction is used to instruct the server to construct the simulation model corresponding to the imaging device. The situation where the imaging device is switched from the non-operational state to the operational state includes a situation where the imaging device is switched from a shutdown state to a startup state.

The initial device state parameter refers to a parameter output by the imaging device based on a digital twin technology, which is used to indicate the initial operational state of the imaging device.

In some embodiments of the present disclosure, by means of outputting the initialization configuration instruction when a switching of the imaging device from the non-operational state to the operational state is detected, the initialization configuration instruction is output to guarantee that the simulation image data corresponding to the imaging device and the working state of the imaging device indicated by the identification content be consist with the actual operational state of the imaging device.

In some embodiments, the server also processes the one or more initial device state parameters to output the initialization configuration instruction when a switch in the operational content of the imaging device is detected. The switching of the operational content includes a situation where the imaging device needs to restart operation. While considering the situation where the imaging device is switched from the non-operational state to the operational state, the situation where an operation content of the imaging device is switched is also considered, to more comprehensively consider scenarios where the initialization configuration instruction is output.

The model initialization configuration parameter refers to the parameter used by the server to construct the simulation model corresponding to the imaging device.

In some embodiments, the server constructs the simulation model based on the one or more model initialization configuration parameters by the digital twin technology. The simulation model is used to generate the simulation image indicating the initial operational state of the imaging device. Some other embodiments of constructing the simulation model based on the digital twin technology may be found in descriptions of FIG. 2.

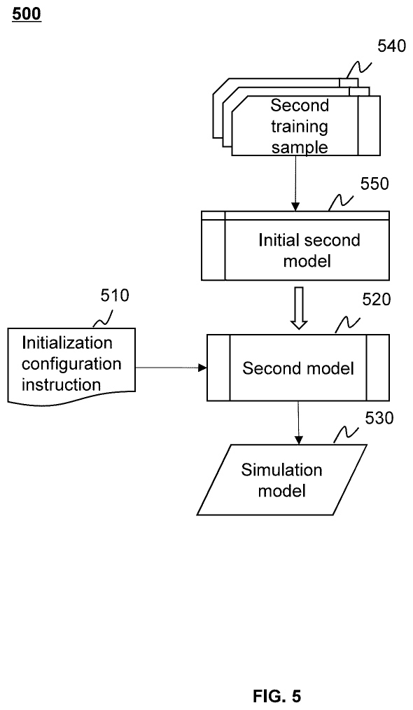

In some embodiments, as shown in FIG. 5, which is a schematic diagram illustrating a process 500 for determining a simulation model based on a second model according to some embodiments of the present disclosure, a server constructs a simulation model 530 through a second model 520 based on an initialization configuration instruction 510.

In some embodiments, the second model 520 is a machine learning model. For example, the second model 520 includes a combination of one or more of a CNN model, a DNN model, etc.

In some embodiments, an input to the second model 520 includes the initialization configuration instruction 510, and an output includes the simulation model 530.

In some embodiments, the second model 520 is obtained by training based on a large number of second training samples 540 with second labels. In some embodiments, the server inputs the second training samples 540 into an initial second model 550, iteratively updates one or more parameters of the initial second model 550 through training until the trained model satisfies a preset termination condition, and then obtains the trained second model 520. The preset termination condition may be that the loss function is less than a threshold or converges, or a training period reaches a threshold. In some embodiments, a method of iteratively updating the one or more parameters of the model includes a random gradient descent, etc.

In some embodiments, the second training samples 540 include sample initialization configuration instructions obtained from historical data. The second labels are actually constructed simulation models from the historical data.

In some embodiments of the present disclosure, generating the simulation model by the second model speeds up the generation of the simulation model, resulting in a more rapid response.

In 420, the simulation image corresponding to the imaging device may be generated using the simulation model.

A process of generating the simulation image using the simulation model is similar to the process of outputting an adjusted simulation image based on an adjusted simulation model, which is not repeated here.

In some embodiments of the present disclosure, by means of generating simulation image data indicating an initial operational state of the imaging device, and an identification content corresponding to the simulation image data based on the initialization configuration instruction issued by the imaging device, when the imaging device is switched from a non-operational state to an operational state, a data consistency between the operational state of the imaging device indicated by the simulation image corresponding to the imaging device and an actual operational state of the imaging device is ensured, which in turn helps to improve an operation efficiency of the imaging device.

It should be noted that the foregoing description of the process of generating the simulation image is for the purpose of exemplification and illustration only and does not limit the scope of application of the present disclosure. For those skilled in the art, various corrections and changes may be made to the process under the guidance of the present disclosure. However, these corrections and changes remain within the scope of the present disclosure.

In some embodiments, the server constructs the simulation model corresponding to the imaging device; generates, based on the simulation model, the simulation image corresponding to the imaging device, the simulation image including the simulation image data of the imaging device; obtains, in real time, in real time, one or more device state parameters of the imaging device; and updates the simulation image in real time based on the one or more device state parameters. The updating the simulation image in real time based on the one or more device state parameters includes: generating, in real time, a module parameter adjustment instruction based on the one or more device state parameters; and updating, in real time, the simulation image based on an adjusted simulation model. Some other embodiments of generating the simulation image corresponding to the imaging device, and updating the simulation image may be seen in descriptions in FIGS. 2-7.

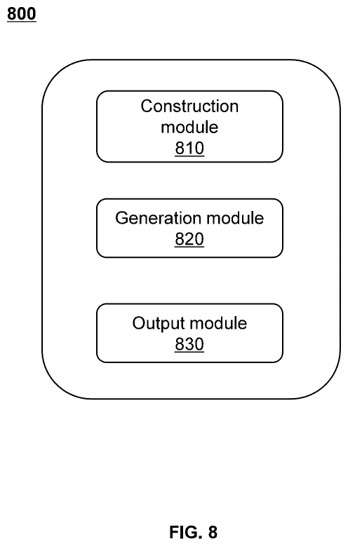

FIG. 8 is a block diagram illustrating an exemplary system for image display in a medical imaging system according to some embodiments of the present disclosure.

In some embodiments, an image display system 800 of a medical image system includes a construction module 810, a generation module 820, and an output module 830.

The construction module 810 is used to construct a simulation model corresponding to the imaging device; the simulation model is used to generate a simulation image corresponding to the imaging device; and the simulation image includes simulation image data of the imaging device. In some embodiments, the simulation image further includes an identification content corresponding to the simulation image data, the identification content includes at least one of a device operation position, a device state parameter change content, a device operation description, or a next operation suggestion.

In some embodiments, the construction module 810 is further used to construct the simulation model corresponding to the imaging device in response to receiving an initialization configuration instruction.

In some embodiments, the construction module 810 is further used to generate one or more model initialization configuration parameters in response to an initialization configuration instruction sent by an imaging device; the one or more model initialization configuration parameters are used to construct the simulation model corresponding to the imaging device. The construction module 810 is further used to generate the simulation image corresponding to the imaging device using the simulation model; the simulation image includes the simulation image data of the imaging device; and the simulation image data is used to be displayed in a viewing device. In some embodiments, the construction module 810 is further used to determine the initialization configuration instruction upon detecting a switch of the imaging device from a non-operational state to an operational state. The initialization configuration instruction is used to instruct a server to construct the corresponding simulation model of the imaging device.

The generation module 820 is used to generate a module parameter adjustment instruction based on one or more device state parameters transmitted by the imaging device. The module parameter adjustment instruction is used to perform corresponding parameter adjustment on the simulation model. In some embodiments, the one or more device state parameters are parameter information corresponding to the device state model output by the imaging device based on a digital twin technology.

In some embodiments, the simulation model includes a plurality of image construction modules; each of the plurality of image construction modules is configured with one or more corresponding construction parameters; and the one or more construction parameters are used to generate the simulation image. The generation module 820 is further used to generate the module parameter adjustment instruction in a situation where the one or more device state parameters transmitted by the imaging device are inconsistent with the one or more construction parameters corresponding to the plurality of image construction modules. The module parameter adjustment instruction is used for instructing the simulation model to adjust the one or more construction parameters corresponding to the plurality of image construction modules.

The output module 830 is used to output the adjusted simulation image based on the adjusted simulation model, and the adjusted simulation image is used for being displayed in the viewing device.

In some embodiments, the viewing device includes at least one of a VR device, an AR device, a hologram device, and a touch display screen. The viewing device is used to display the simulation image matching a first display mode based on a received first display mode configuration instruction. In some embodiments, the viewing device is further used to display the simulation image matching a second display mode based on a received second display mode configuration instruction.

In some embodiments, the output module 830 is further used to output simulation image adjustment information in response to receiving an interactive instruction, the simulation image adjustment information being configured for instructing the server to adjust the simulation model. In some embodiments, the output module 830 is further used to output one or more device state adjustment parameters based on the simulation image adjustment information, the one or more device state adjustment parameters being configured for instructing the imaging device to make a corresponding operational state adjustment.

In some embodiments, the output module 830 is further used to display the simulation image data corresponding to the simulation image transmitted by the server, and the identification content corresponding to the simulation image data in a situation where the first display mode is a guidance mode; and display the simulation image data corresponding to the simulation image transmitted by the server in a situation where the first display mode is a consultation mode.

In some embodiments, the output module 830 is further used to display the simulation image data corresponding to the simulation image transmitted by the server in a situation where the second display mode is a default display mode; in a situation where the second display mode is a partial display mode, display the simulation image data corresponding to the simulation image transmitted by the server, and display, based on a simulation image data selection instruction, a portion of the identification content corresponding to selected simulation image data; and in a situation where the second display mode is a full display mode, display the simulation image data corresponding to the simulation image transmitted by the server, and the identification content corresponding to the simulation image data.

In some embodiments, the imaging device is a handheld ultrasound device.

See FIGS. 2-7 and their related descriptions for more descriptions of the construction module 810, the generation module 820, and the output module 830.

It should be understood that the system and the modules shown in FIG. 8 are implemented in various manners. It should be noted that the above description of the modules of the system for image display in a medical imaging system is for descriptive convenience only, and it does not limit the present disclosure to the scope of the embodiments cited. It is to be understood that for those skilled in the art, after understanding the principle of the system, individual modules are arbitrarily combined, or form a subsystem to connect with other modules without departing from this principle. In some embodiments, the construction module 810, the generation module 820, and the output module 830 disclosed in FIG. 8 are different modules in a single system, or a single module realizes functions of two or more modules. For example, the individual modules share one storage module, or the individual modules each have a respective storage module. Such deformations are within the scope of protection of the present disclosure.

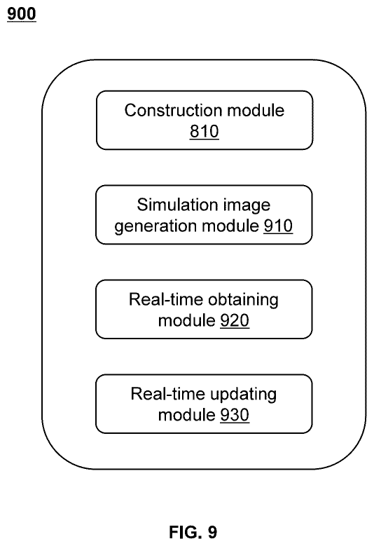

FIG. 9 is a block diagram illustrating an exemplary system for image display in a medical imaging system according to some other embodiments of the present disclosure.

In some embodiments, an image display system 900 of a medical image system includes the construction module 810, a simulation image generation module 910, a real-time obtaining module 920, and a real-time updating module 930.

The construction module 810 is used to construct a simulation model corresponding to an imaging device. In some embodiments, the construction module 810 is further used to construct the simulation model corresponding to the imaging device in response to receiving an initialization configuration instruction.

The simulation image generation module 910 is used to generate a simulation image corresponding to the imaging device based on the simulation model, the simulation image including simulation image data of the imaging device. In some embodiments, the simulation image further includes an identification content corresponding to the simulation image data, the identification content includes at least one of a device operation position, a device state parameter change content, a device operation description, or a next operation suggestion.

The real-time obtaining module 920 is used to obtain, in real time, one or more device state parameters of the imaging device.

The real-time updating module 930 is used to update, in real-time, the simulation image based on the one or more device state parameters. In some embodiments, the real-time updating module 930 is further used to generate, in real time, a module parameter adjustment instruction based on the one or more device state parameters, the module parameter adjustment instruction being used for adjusting one or more parameters of the simulation model. The real-time updating module 930 is further used to update, in real time, the simulation image based on an adjusted simulation model.

More descriptions of the construction module 810, the simulation image generation module 910, the real-time obtaining module 920, and the real-time updating module 930 may be found in FIGS. 2-7 and their related descriptions.

It should be understood that the system and its modules shown in FIG. 9 are realized in various manners. It should be noted that the above description of the modules of the system for image display in a medical imaging system is for descriptive convenience only, and it does not limit the present disclosure to the scope of the embodiments cited. It is to be understood that for those skilled in the art, after understanding the principle of the system, individual modules are arbitrarily combines or a subsystem is formed to connect with other modules without departing from this principle. In some embodiments, the construction module 810, the simulation image generation module 910, the real-time obtaining module 920, and the real-time updating module 930 disclosed in FIG. 9 are different modules in a system or a single module that realizes the functions of two or more of the above-described modules. For example, the individual modules share one storage module, or the individual modules each have a respective storage module. Such deformations are within the scope of protection of the present disclosure.

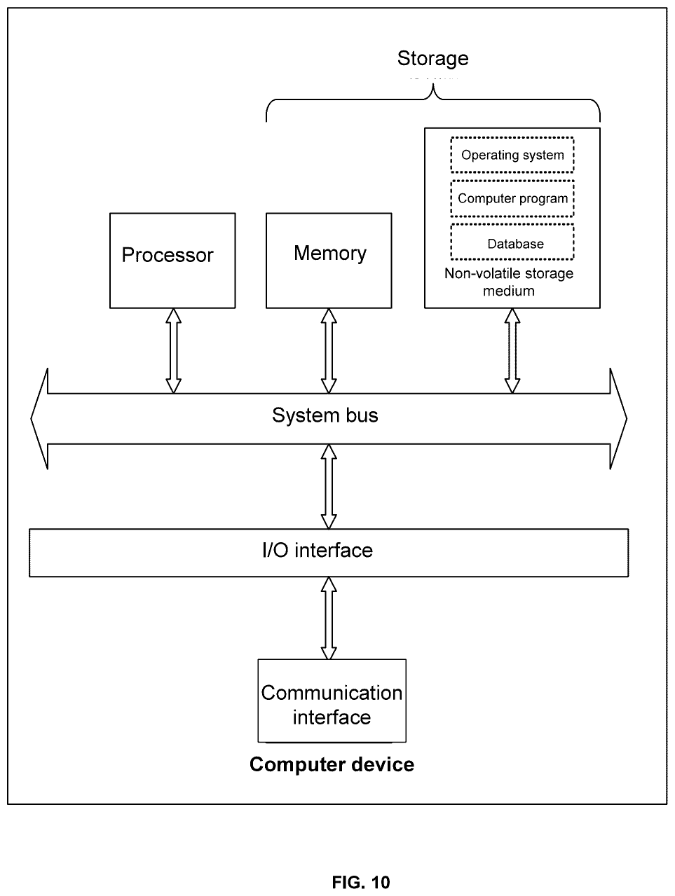

In one embodiment, a computer device is provided, which is a server, and an internal structure diagram thereof is shown in FIG. 10. The computer device includes a processor, a storage, an input/output (I/O) interface, and a communication interface. The processor, the storage, and the I/O interface are connected through a system bus, and the communication interface is connected to the system bus through the input/output interface. The processor of the computer device is used to provide calculating and controlling capabilities. The storage of the computer device includes a non-volatile storage medium and a memory. The non-volatile storage medium stores an operating system, a computer program, and a database. The memory provides an environment for running of the operating system and the computer program in the non-volatile storage medium. The database of the computer device is used for storing data such as data related to the simulation image of the imaging device. The input/output interface of the computer device is used for exchanging information between the processor and an external device. The communication interface of the computer device is used to communicate with an external terminal through a network connection. When executed by a processor, the computer program implements the method for image display in a medical imaging system.

Those skilled in the art may understand that the structure illustrated in FIG. 10 is only a block diagram of a part of the structure related to the program of the present disclosure, which does not constitute a limitation of the computer device where the program of the present disclosure is applied on, and the specific computer device includes more or fewer components than shown in the drawings, or combines certain components, or has a different arrangement of the components.

In some embodiments, a computer-readable storage medium stores computer instructions, and when the computer reads the computer instructions in the storage medium, the computer runs the method for image display in a medical imaging system.

Beneficial effects brought about by embodiments of the present disclosure include, but are not limited to: (1) a real-time operation guidance is provided to the user by displaying the simulation image including the identification content corresponding to the simulation image data in front of user's eyes through the viewing device. This enables the user to obtain in real time the current device operation position, the device state parameter change content, the device operation instruction, the next operation suggestion, etc., to help the user better operate the imaging device, with a strong interactivity. (2) The first model allows for a fast and accurate prediction of the one or more current device state parameters, which in turn allows for generating a module parameter adjustment instruction based on the predicted current device state parameters when a network transmission quality is poor (or even when the data transmission is interrupted), so as to ensure that an image display is not damaged. (3) By means of adjusting the corresponding parameters of each image construction module in the simulation model corresponding to the imaging device based on the one or more device state parameters transmitted by the imaging device, a data consistency between the operational state of the imaging device indicated by the simulation image corresponding to the imaging device and the actual operational state of the imaging device is ensured, thereby ensuring that a user of the viewing device is able to accurately informed of the operational state of the imaging device. And, by dividing the simulation model into a count of image construction modules and analyzing the consistency between the one or more device state parameters and the one or more construction parameters corresponding to each of the image construction modules, and in case of inconsistency, adjustments may be made to the inconsistent image construction modules without having to adjust all the modules, which reduces a calculation pressure on the server when processing data and improves the efficiency. (4) By constructing the simulation model with a real-time data synchronization function for generating the simulation image indicating the current operational state of the imaging device by adopting a digital twin technology between the server and the imaging device, the real-time display of the simulation image data indicating the current operational state of the imaging device as well as the identification content corresponding to the simulation image data in the viewing device are implemented, which not only solves the problem that an operator of the imaging device is prone to mis-operation due to the difficulty of accurately knowing the operational state of the imaging device, but also effectively improves the operation efficiency of the imaging device. (5) By performing corresponding adjustment on the simulation image generated by the simulation model corresponding to the imaging device and the actual operational state of the imaging device based on the interactive instruction issued by the user, not only the data consistency between the operational state of the imaging device indicated by the simulation image corresponding to the imaging device and the actual operational state of the imaging device is ensured, but also effectively improves the operational efficiency of the imaging device. (6) By means of outputting the initialization configuration instruction when a switching of the imaging device from the non-operational state to the operational state is detected, the initialization configuration instruction is output to guarantee that the simulation image data corresponding to the imaging device and the working state of the imaging device indicated by the identification content consist with the operational state in which the imaging device is actually in. (7) By means of generating simulation image data indicating an initial operational state of the imaging device, and an identification content corresponding to the simulation image data based on the initialization configuration instruction issued by the imaging device, when the imaging device is switched from a non-operational state to an operational state, a data consistency between the operational state of the imaging device indicated by the simulation image corresponding to the imaging device and an actual operational state of the imaging device is ensured, which in turn helps to improve an operation efficiency of the imaging device.

It should be noted that the user information (including, but not limited to, user device information, user personal information, etc.) and the data (including, but not limited to, data used for analysis, data stored, data displayed, etc.) involved in the present disclosure are information and data authorized by the user or fully authorized by the parties, and the collection, use and processing of the relevant data need to comply with the relevant laws and regulations and standards of the relevant countries and regions.

Those skilled in the art may understand that all or part of the processes in the methods of the above embodiments are implemented by means of a computer program to instruct the relevant hardware to complete, and that the computer program may be stored in a non-volatile computer-readable storage medium, when the computer program is executed, the processes of the above-described methods are included. Any reference to a memory, database, or other medium used in the embodiments provided in the present disclosure may include at least one of a non-volatile and a volatile memory. The non-volatile memory may include a Read-Only Memory (ROM), a magnetic tape, a floppy disk, a flash memory, an optical memory, a high-density embedded non-volatile memory, a ReRAM, a magneto resistive random access memory (MRAM), a ferroelectric random access memory (FRAM), a phase change memory (PCM), a graphene memory, etc. The volatile memory may include a random access memory (RAM) or an external cache memory, etc. As an illustration and not a limitation, the RAM may be in various forms, such as a static random access memory (SRAM) or a dynamic random access memory (DRAM), etc. The database involved in embodiments provided in the present disclosure may include at least one of a relational database and a non-relational database. The non-relational database may include but not limited to a blockchain-based distributed database, etc. The processor involved in the embodiments of the present disclosure may be a universal processor, a central processor, a graphics processor, a digital signal processor, a programmable logic device, a quantum computing based data processing logic device, etc., which is not limited here.

The various technical features of the above embodiments are combined in any combinations, and for a conciseness of the description, all possible combinations of the various technical features in the above embodiments are not described, however, as long as there is no contradiction in the combinations of these technical features, these combinations should be considered to be within the scope of the present disclosure.

The above-described embodiments express only several embodiments of the present disclosure, which are described in a more specific and detailed manner, but are not to be construed as a limitation on the scope of the patent of the present disclosure. It should be noted that, for those skilled in the art, several deformations and improvements may be made without departing from the conception of the present disclosure, and all of the deformations and improvements fall within the scope of protection of the present disclosure. Thus, the scope of protection of the present disclosure shall be subject to the appended claims.

1. A method implemented on at least one machine each of which has at least one processor and at least one storage device in a server of a medical image system for image display in a medical imaging system, wherein the medical image system includes an imaging device and a viewing device both connected to the server, and the method comprises:
constructing a simulation model corresponding to the imaging device, the simulation model being configured to generate a simulation image corresponding to the imaging device, and the simulation image including simulation image data of the imaging device;
generating a module parameter adjustment instruction based on one or more device state parameters transmitted by the imaging device, the module parameter adjustment instruction being configured to adjust one or more parameters of the simulation model; and
outputting an adjusted simulation image based on an adjusted simulation model, the adjusted simulation image being used for displaying in the viewing device.

2. The method of claim 1, wherein the constructing a simulation model corresponding to the imaging device comprises:
in response to receiving an initialization configuration instruction, constructing the simulation model corresponding to the imaging device.

3. The method of claim 1, wherein the simulation image further includes an identification content corresponding to the simulation image data, the identification content includes at least one of a device operation position, a device state parameter change content, a device operation description, or a next operation suggestion.

4. The method of claim 1, wherein the simulation model includes a plurality of image constructing modules, each of the plurality of image constructing modules is configured with one or more construction parameters, the one or more construction parameters are used for generating the simulation image, and
the generating a module parameter adjustment instruction based on one or more device state parameters transmitted by the imaging device comprises:
generating the module parameter adjustment instruction in a situation where the one or more device state parameters transmitted by the imaging device are inconsistent with the one or more construction parameters corresponding to the each of the plurality of image construction modules; wherein the module parameter adjustment instruction is used for instructing the simulation model to adjust the one or more construction parameters corresponding to the each of the plurality of image construction modules.

5. The method of claim 2, wherein the in response to receiving an initialization configuration instruction, constructing the simulation model corresponding to the imaging device comprises:
in response to the initialization configuration instruction sent by the imaging device, generating one or more model initialization configuration parameters, the one or more model initialization configuration parameters being configured for constructing the simulation model corresponding to the imaging device; and
generating the simulation image corresponding to the imaging device using the simulation model, the simulation image including the simulation image data of the imaging device, and the simulation image data being used for displaying in the viewing device.

6. The method of claim 1, wherein the one or more device state parameters are outputted by the imaging device based on a digital twin technology.

7. The method of claim 2, wherein the initialization configuration instruction is determined by:
determining the initialization configuration instruction in a situation where the imaging device is detected to be switched from a non-operational state to an operational state, the initialization configuration instruction being configured for instructing the server to construct a simulation model corresponding to the imaging device.

8. The method of claim 1, further comprising:
in response to receiving an interactive instruction, outputting simulation image adjustment information, the simulation image adjustment information being configured for instructing the server to adjust the simulation model.

9. The method of claim 8, further comprising:
outputting one or more device state adjustment parameters based on the simulation image adjustment information, the one or more device state adjustment parameters being configured for instructing the imaging device to make a corresponding operational state adjustment.

10. The method of claim 1, wherein:
the viewing device includes at least one of a visual reality (VR) device, an augmented reality (AR) device, a holographic imaging device, or a touch display screen; and
the viewing device is configured to display the simulation image matching a first display mode based on a received first display mode configuration instruction.

11. The method of claim 10, wherein the simulation image matching the first display mode is displayed by:
in a situation where the first display mode is a guidance mode, displaying the simulation image data corresponding to the simulation image transmitted by the server, and the identification content corresponding to the simulation image data; and
in a situation where the first display mode is a consultation mode, displaying the simulation image data corresponding to the simulation image transmitted by the server.

12. The method of claim 1, wherein the viewing device is further configured to display the simulation image matching a second display mode based on a received second display mode configuration instruction.

13. The method of claim 12, wherein the displaying the simulation image matching the second display mode is displayed by:
in a situation where the second display mode is a default display mode, displaying the simulation image data corresponding to the simulation image transmitted by the server;
in a situation where the second display mode is a partial display mode, displaying the simulation image data corresponding to the simulation image transmitted by the server, and displaying, based on a simulation image data selection instruction, a portion of the identification content corresponding to selected simulation image data; and
in a situation where the second display mode is a full display mode, displaying the simulation image data corresponding to the simulation image transmitted by the server, and the identification content corresponding to the simulation image data.

14. The method of claim 1, wherein the imaging device is a handheld ultrasound device.

15. A method implemented on at least one machine each of which has at least one processor and at least one storage device in a server of a medical image system for image display in a medical imaging system, wherein the medical image system includes an imaging device and a viewing device both connected to the server, and the method comprises:
constructing a simulation model corresponding to the imaging device;
generating a simulation image corresponding to the imaging device based on the simulation model, wherein the simulation image includes simulation image data of the imaging device;
obtaining, in real time, one or more device state parameters of the imaging device; and
updating the simulation image in real time based on the one or more device state parameters.

16. The method of claim 15, wherein the constructing a simulation model corresponding to the imaging device comprises:
in response to receiving an initialization configuration instruction, constructing the simulation model corresponding to the imaging device.

17. The method of claim 15, wherein the simulation image further includes identification content corresponding to the simulation image data, and the identification content includes at least one of a device operation position, a device state parameter change content, a device operation description, or a next operation suggestion.

18. The method of claim 15, wherein the updating the simulation image in real time based on the one or more device state parameters comprises:
generating, in real time, a module parameter adjustment instruction based on the one or more device state parameters, the module parameter adjustment instruction being used for adjusting one or more parameters of the simulation model; and
updating, in real time, the simulation image based on an adjusted simulation model.

19-24. (canceled)

25. A non-transitory computer-readable storage medium storing computer instructions, wherein when reading the computer instructions in the storage medium, a computer implements the method for image display in a medical imaging system, wherein the medical image system includes an imaging device and a viewing device both connected to the server, and the method comprises:
constructing a simulation model corresponding to the imaging device, the simulation model being configured to generate a simulation image corresponding to the imaging device, and the simulation image including simulation image data of the imaging device;
generating a module parameter adjustment instruction based on one or more device state parameters transmitted by the imaging device, the module parameter adjustment instruction being configured to adjust one or more parameters of the simulation model; and
outputting an adjusted simulation image based on an adjusted simulation model, the adjusted simulation image being used for displaying in the viewing device.

26. The method of claim 4, wherein the generating a module parameter adjustment instruction based on one or more device state parameters transmitted by the imaging device further comprises:
predicting, based on a device state parameter sequence transmitted by the imaging device and received during a preset period of time, one or more current device state parameters transmitted by the imaging device; and
in response to that the one or more current device state parameters transmitted by the imaging device are inconsistent with the one or more construction parameters corresponding to the plurality of image construction modules, generating the module parameter adjustment instruction by processing the one or more current device state parameters.
